(19) United States
(12) Patent Application Publication
(10) Pub. No.: US 2006/0030591 A1
(43) Pub. Date: Feb. 9, 2006

(54) Crystalline polymorph of pipindoxifene hydrochloride monohydrate

(75) Inventors: Christopher Demerson, Kirkland (CA); Gloria Cheal, Beaconsfield (CA)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/197,643

(22) Filed: Aug. 4, 2005

Related U.S. Application Data
(60) Provisional application No. 60/599,825, filed on Aug. 5, 2004.

(51) Int. Cl. A61K 31/454; C07D 403/02

(52) U.S. Cl. 514/323; 546/201

(57) ABSTRACT

The present invention is directed to a crystalline polymorph of pipindoxifene hydrochloride monohydrate, compositions containing the same, preparations thereof, and uses thereof.

### CROSS REFERENCE TO RELATED APPLICATIONS

This application claims the benefit of U.S. Ser. No. 60/599,825, filed Aug. 5, 2004, the disclosure of which is incorporated herein by reference in its entirety.

### FIELD OF THE INVENTION

The present invention relates to a crystalline polymorph, designated form I, of the selective estrogen receptor modulator, 2-(4-hydroxyphenyl)-3-methyl-1-[4-(2-piperidin-1-yl-ethoxy)-benzyl]-1H-indol-5-ol hydrochloride (pipindoxifene hydrochloride).

### BACKGROUND OF THE INVENTION

Pipindoxifene hydrochloride, (2-(4-hydroxyphenyl)-3-methyl-1-[4-(2-piperidin-1-ylethoxy)-benzyl]-1H-indol-5-ol hydrochloride) has the chemical formula as shown below.

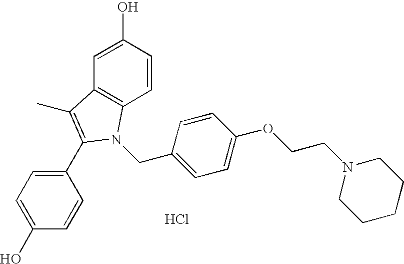

The compound belongs to the class of drugs typically referred to as selective estrogen receptor modulators (SERMs). Consistent with its classification, pipindoxifene demonstrates affinity for estrogen receptors (ER) but shows tissue selective estrogenic effects, such as little or no uterotropic activity.

Pipindoxifene is a variant of zinidoxifene and ZK119010 (Von Angerer, et al., J. Med. Chem. 33:2635-2640 (1990) and Von Angerer, et al., J. Med. Chem. (1984) 27:1439-1447). It has a rigidified alkylamino side chain compared to ZK119010 to provide optimized binding to helix 12 of the ligand binding domain of estrogen receptor.

The complexity of the estrogen receptor (ER) in its interaction with ligands, agonists and antagonists is well known. Thus, the search for drugs that provide therapeutic promise for the treatment of cancers known to involve estrogen receptor function and dysfunction is a challenge. One of the most commonly prescribed estrogen-receptor blockers for the treatment of breast cancer is tamoxifen. In recent preclinical studies pipindoxifene inhibited the growth of tamoxifen-resistent MCF-7 breast cancer xenografts. Other studies show effectiveness of pipindoxifene in tamoxifen-sensitive cells lines as well. When compared to both tamoxifen and raloxifene, pipindoxifene has demonstated an improved profile in preclinical studies.

The method for the synthesis of pipindoxifene hydrochloride is detailed in Miller, et al., J. Med. Chem. (2001) 44:1654-1657, which is incorporated by reference herein. The 3-methyl indole core is synthesized from a-bromopropiophenone and aniline hydrochloride via a Bischler-type indole synthesis, Von Angerer, et al., J. Med. Chem. (1984) 27:1439-1447. The side chain is prepared by alkylation of 4-OH benzyl alcohol with ethyl bromoacetate followed by conversion of the alcohol to benzyl chloride with SOCl₂ in THF. The reaction of the indole with the side chain occurs in the presence of sodium hydride in dimethylformamide. The ester is then reduced with LAH and the primary alcohol is converted to the corresponding bromide with carbon tetrabromide and triphenylphosphine. Subsequent steps include substituting the bromide with piperidine, hydrogenation and conversion to the hydrochloride salt. The HCl salt prepared by the above method results in white crystalline monohydrate (Karl Fisher analysis: 3.52%; 3.23% found) product having a relatively broad melting point range from 185.3° C. to 186.6° C. A similar procedure in U.S. Pat. No. 5,998,402 was used to make crystalline pipindoxifene hydrochloride monohydrate with a melting point of 184-185° C. (and 177-182° C. for a second crop). Alternative preparations of pipindoxifene hydrochloride and related compounds are reported in U.S. Pat. Nos. 6,268,504 and 6,242,605.

The crystalline polymorph form of a particular drug is often an important determinant of the drug's ease of preparation, stability, solubility, storage stability, ease of formulation and in vivo pharmacology. Polymorphic forms occur where the same composition of matter crystallizes in a different lattice arrangement resulting in different thermodynamic properties and stabilities specific to the particular polymorph form. In cases where two or more polymorph substances can be produced, it is desirable to have a method to make both polymorphs in pure form. In deciding which polymorph is preferable, the numerous properties of the polymorphs must be compared and the preferred polymorph chosen based on the many physical property variables. It is entirely possible that one polymorph form can be preferable in some circumstances where certain aspects such as ease of preparation, stability, etc are deemed to be critical. In other situations, a different polymorph maybe preferred for greater solubility and/or superior pharmacokinetics.

Because improved drug formulations, showing, for example, better bioavailability or better stability are consistently sought, there is an ongoing need for new or purer polymorphic forms of existing drug molecules. The polymorph of pipindoxifene hydrochloride described herein helps meet these and other needs.

### BRIEF DESCRIPTION OF THE DRAWINGS

FIG. 1 depicts a powder X-ray diffraction pattern of pipindoxifene hydrochloride monohydrate form II polymorph, where the diffraction angle (20) ranges from 0-40 degrees with a step of 2.5 degrees.

FIG. 2 depicts a powder X-ray diffraction pattern of pipindoxifene hydrochloride monohydrate form I polymorph, where the diffraction angle (20) ranges from 0-40 degrees with a step of 2.5 degrees.

FIG. 3 depicts a differential scanning calorimetric (DSC) trace of a mixture of pipindoxifene hydrochloride monohydrate form II and form I.

FIG. 4 depicts a differential scanning calorimetric (DSC) trace of pipindoxifene hydrochloride monohydrate form I.

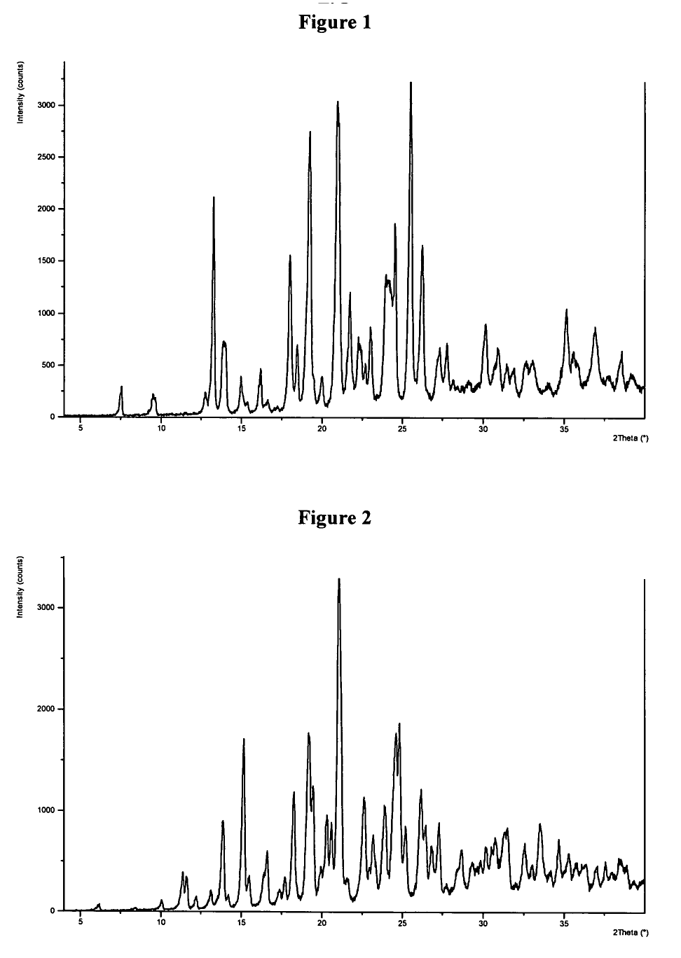

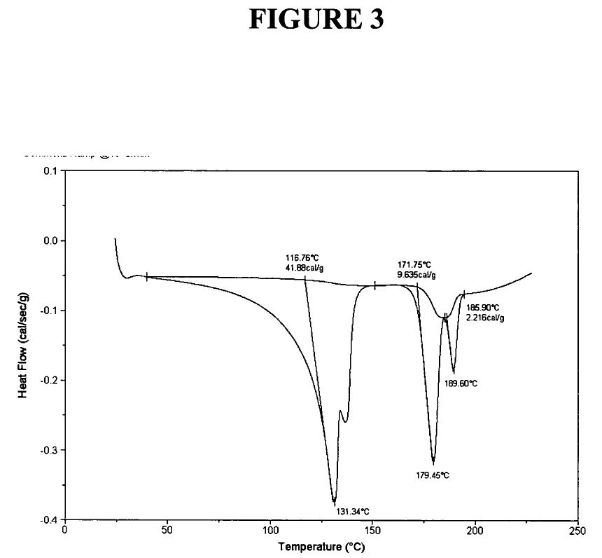

### SUMMARY OF THE INVENTION

The present invention provides a crystalline polymorph (form I) of pipindoxifene hydrochloride monohydrate characterized by XRPD and DSC.

The present invention further provides compositions containing the polymorph of the invention.

The present invention further provides methods of preparing pipindoxifene hydrochloride monohydrate polymorphic form I comprising dissolving pipindoxifene hydrochloride in a solvent mixture comprising an alcohol, water, and optionally an ether; and precipitating the form I from the solvent mixture.

The present invention further provides methods of preparing the polymorphic form I by recrystallizing pipindoxifene hydrochloride monohydrate form II from a solvent mixture comprising water and ethanol, wherein the volume ratio of water to alcohol is less than about 1:5.

The present invention further provides methods of treating a mammal having a disease or syndrome associated with estrogen deficiency or excess of estrogen comprising administering to the mammal a therapeutically effective amount of a polymorph of the invention.

The present invention further provides methods of treating a mammal having a disease or disorder associated with proliferation or abnormal development of mammary tissues comprising administering to the mammal a therapeutically effective amount of a polymorph of the invention.

The present invention further provides methods of lowering cholesterol in a mammal comprising administering to the mammal a therapeutically effective amount of a polymorph of the invention.

The present invention further provides methods of inhibiting bone loss in a mammal comprising administering to the mammal a therapeutically effective amount of a polymorph the invention.

The present invention further provides methods of treating breast cancer in a mammal comprising administering to the mammal a therapeutically effective amount of a polymorph of the invention.

The present invention further provides methods of treating a postmenopausal woman for one or more vasomotor disturbances comprising administering to the postmenopausal woman a therapeutically effective amount of a polymorph of the invention.

### DETAILED DESCRIPTION

The present invention provides a crystalline polymorph of pipindoxifene hydrochloride hydrate, referred to herein as form I, which can be identified by one or more solid state analytical methods. For example, form I can be identified by its powder X-ray diffraction pattern which is provided in FIG. 2. Powder X-ray diffraction data consistent with form I is provided in Table 1 below.

TABLE 1

| | Intensity, |
|---|---|
| Degree (2θ) | Counts Per Second (CPS) |
| 11.8 | 460 |
| 13.9 | 970 |
| 15.1 | 1750 |
| 16.6 | 590 |
| 18.1 | 1240 |
| 19.2 | 1760 |
| 20.4 | 890 |
| 20.7 | 860 |
| 21.2 | 3400 |
| 22.5 | 1190 |
| 23.1 | 740 |
| 24.3 | 1760 |
| 25.0 | 800 |
| 26.4 | 1070 |
| 26.9 | 810 |
| 28.8 | 700 |
| 30.2 | 720 |
| 30.9 | 770 |
| 31.8 | 830 |
| 33.2 | 860 |
| 34.6 | 840 |

In some embodiments, the crystalline polymorph (form I) of pipindoxifene hydrochloride is characterized by a powder X-ray diffraction pattern having characteristic peaks, in terms of 20, at about 21.2° and about 24.3°. In some embodiments, characteristic peaks at about 15.1° and about 19.2° are further present. In further embodiments, the powder X-ray diffraction pattern further includes at least 5 characteristic peaks, in terms of 2θ, selected from about 13.9°, about 15.1°, about 18.1°, about 19.2°, about 21.2°, about 22.5°, about 24.3°, and about 26.4°. In yet further embodiments, form I is characterized by a powder X-ray diffraction pattern substantially as shown in FIG. 2. With respect to the term “substantially,” one skilled in the art would understand that the relative intensities of the peaks can vary, depending upon the sample preparation technique, the sample mounting procedure and the particular instrument employed. Moreover, instrument variation and other factors can affect the 2-theta values. Therefore, the XRPD peak assignments can vary by plus or minus about 0.2°.

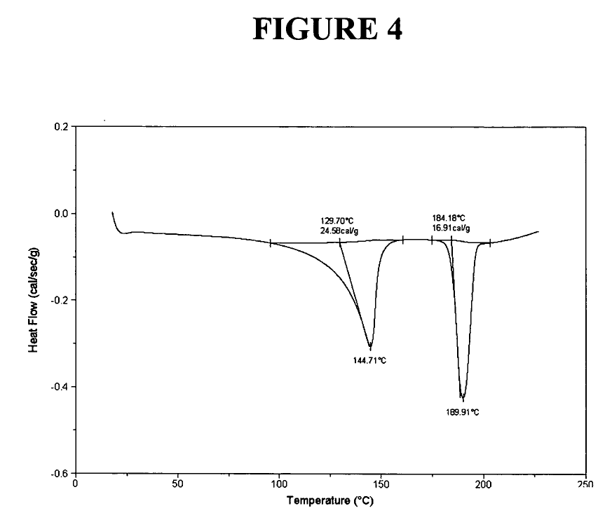

Pipindoxifene form I can also be identified by its characteristic differential scanning (DSC) trace such as shown in FIG. 4. In some embodiments, form I is characterized by a DSC trace showing maxima at about 145 and 190° C. The lower temperature peak likely corresponds to a dehydration event. The higher temperature peak is believed to correspond to a melting endotherm. For DSC, it is known that the temperatures observed will depend upon the rate of temperature change as well as sample preparation technique and the particular instrument employed. Thus, the values reported herein relating to DSC thermograms can vary by plus or minus about 4° C.

Pipindoxifene hydrochloride hydrate can also exist as a second polymorph designated form II. Sample data for certain physical properties are compared for form I and form II polymorphs below in Table 2.

TABLE 2

| Measurement | Form I | Form II |
|---|---|---|
| DSC | Two Endotherms | Two Endotherms |
| | 145° C. and 190° C. | 131° C. and 179° C. |

As can be seen in Table 2, the two crystalline polymorphs have discernable physical and spectroscopic characteristics. Form I appears to be thermodynamically more stable than form II, and would therefore be expected to exhibit superior stability which is often desirable in the preparation of pharmaceutical formulations. Less thermodynamically stable form II would be expected to possess higher solubility which could contribute to improved bioavailability and uptake.

Examples of preparations of forms I and II are provided in the Examples. In general, form I can be prepared by dissolving pipindoxifene hydrochloride (any form, including amorphous) in a suitable solvent containing water and crystallizing the polymorph product from the solvent by any of numerous routine methods in the art such as by cooling or evaporating the solvent to induce precipitation. Suitable solvents include a mixture of water, an alcohol, and optionally an ether. Water content of the solvent appears to influence the relative amounts of form I and form II which precipitate. Higher amounts of water in the solvent tend to favor form II while lower amounts of water tend to favor form I.

In preparations of form II, the volume ratio of water to alcohol in the crystallizing solvent can be greater than about 1:5. In some embodiments, the volume ratio of water to alcohol in preparations of form II is about 2 to about 1:5, about 1 to about 1:5, about 1:2 to about 1:5, about 2:5 to about 1:5, about 1:3 to about 1:5, or about 2:5.

In preparations of form I, the volume ratio of water to alcohol in the crystallizing solvent can be less than about 1:5. In some embodiments, the volume ratio of water to alcohol in preparations of form I is about 1:5 to about 1:50, about 1:5 to about 1:20, about 1:5 to about 1:10, or about 1:7. In some embodiments, the crystallizing solvent contains water and ethanol. In some embodiments, the crystallizing solvent contains water, ethanol and tetrahydrofuran.

Suitable alcohols include methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol. In some embodiments, the alcohol is ethanol.

Suitable ethers include dimethoxymethane, tetrahydrofuran, 1,3-dioxane, 1,4-dioxane, furan, diethyl ether, ethylene glycol dimethyl ether, ethylene glycol diethyl ether, diethylene glycol dimethyl ether, diethylene glycol diethyl ether, triethylene glycol dimethyl ether, anisole, or t-butyl methyl ether. In some embodiments, the ether solvent is tetrahydrofuran.

The methods for preparation of form I provided herein can result in substantially pure form I (e.g., compositions containing less than about 10%, about 5%, or about 3% of form I by weight) as well as mixtures enriched in form I (e.g., greater than about 50% form I relative to form II by weight). Accordingly, the present invention further provides compositions containing form I. In some embodiments, at least about 50%, about 70%, about 80%, about 90%, about 95%, about 97%, or about 98.0%, about 98.1%, about 98.2%, about 98.3%, about 98.4%, about 98.5%, about 98.6%, about 98.7%, about 98.8%, about 98.9%, about 99.0%, about 99.1%, about 99.2%, about 99.3%, about 99.4%, about 99.5%, about 99.6%, about 99.7%, about 99.8%, or about 99.9% by weight of total pipindoxifene hydrochloride monohydrate in a composition is present as form I. In further embodiments, compositions of the present invention consist essentially of pipindoxifene hydrochloride monohydrate where at least about 95%, about 97%, about 98.0%, about 98.1%, about 98.2%, about 98.3%, about 98.4%, about 98.5%, about 98.6%, about 98.7%, about 98.8%, about 98.9%, about 99.0%, about 99.1%, about 99.2%, about 99.3%, about 99.4%, about 99.5%, about 99.6%, about 99.7%, about 99.8%, or about 99.9% by weight of the pipindoxifene hydrochloride monohydrate is present in the composition as form I. In some embodiments, the remainder pipindoxifene hydrochloride is present as form II or as amorphous material. Respective amounts of polymorphic forms of pipindoxifene hydrochloride in a composition can be determined by any suitable spectroscopic method, such as X-ray powder diffraction or DSC.

As described in Greenberger, et al., Clin. Cancer Res. (2001) 7:3166-3177, pipindoxifene and salts thereof are selective estrogen agonists with affinity for the estrogen receptor. Unlike other types of estrogen agonists, pipindoxifene and salts thereof are antiestrogenic in the uterus and can antagonize the trophic effects of estrogen agonists in uterine tissues. Accordingly, polymorphs of pipindoxifene hydrochloride and compositions containing the same can find many uses related to treating disease states or syndromes associated with an estrogen deficiency or an excess of estrogen. The polymorph can also be used in methods of treatment for diseases or disorders which result from proliferation or abnormal development, actions or growth of endometrial or endometrial-like tissues.

The present polymorphic form of pipindoxifene hydrochloride has the ability to behave like an estrogen agonist by lowering cholesterol and inhibiting bone loss. Accordingly, the polymorph is useful for treating many maladies which result from estrogen effects and estrogen excess or deficiency including osteoporosis, prostatic hypertrophy, male pattern baldness, vaginal and skin atrophy, acne, dysfunctional uterine bleeding, endometrial polyps, benign breast disease, uterine leiomyomas, adenomyosis, ovarian cancer, infertility, breast cancer, endometriosis, endometrial cancer, polycystic ovary syndrome, cardiovascular disease, contraception, Alzheimer's disease, cognitive decline and other CNS disorders, as well as certain cancers including melanoma, prostrate cancer, cancers of the colon, CNS cancers, among others. Additionally, these polymoprhs can be used for contraception in pre-menopausal women, as well as hormone replacement therapy in post-menopausal women (such as for treating vasomotor disturbances such as hot flush) or in other estrogen deficiency states where estrogen supplementation would be beneficial. It can also be used in disease states where amenorrhea is advantageous, such as leukemia, endometrial ablations, chronic renal or hepatic disease or coagulation diseases or disorders.

The polymorph of the invention can also be used in methods of inhibiting bone loss. Bone loss often results from an imbalance in an individual's formation of new bone tissues and the resorption of older tissues, leading to a net loss of bone. Such bone depletion results in a range of individuals, particularly in post-menopausal women, women who have undergone bilateral oophorectomy, those receiving or who have received extended corticosteroid therapies, those experiencing gonadal dysgenesis, and those suffering from Cushing's syndrome. Special needs for bone, including teeth and oral bone, replacement can also be addressed using these polymorphs in individuals with bone fractures, defective bone structures, and those receiving bone-related surgeries and/or the implantation of prosthesis. In addition to the problems described above, the polymorph can be used in treatments for osteoarthritis, hypocalcemia, hypercalcemia, Paget's disease, osteomalacia, osteohalisteresis, multiple myeloma and other forms of cancer having deleterious effects on bone tissues.

Methods of treating the diseases and syndromes listed herein are understood to involve administering to an individual in need of such treatment a therapeutically effective amount of the polymorph of the invention, or composition containing the same. As used herein, the term “treating” in reference to a disease is meant to refer to preventing, inhibiting and/or ameliorating the disease.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and
(3) ameliorating the disease; for example, ameliorating a disease, condition or disorder in an individual that is experiencing or displaying the pathology or symptomatology of the disease, condition or disorder (i.e., reversing the pathology and/or symptomatology).

The invention also includes pharmaceutical compositions utilizing one or more of the present polymorphs along with one or more pharmaceutically acceptable carriers, excipients, etc.

Formulations of pipindoxifene hydrochloride monohydrate form I include therapeutically effective amounts that can be given in daily doses ranging from 0.1 mg to 1000 mg to a person in need. Example dose ranges vary from 10 mg/day to about 600 mg/day or from 10 mg/day to about 60 mg/day. The dosing can be either in a single dose or two or more divided doses per day. Such doses can be administered in any manner that facilitates the compound's entry into the bloodstream including orally, via implants, parenterally (including intravenous, intraperitoneal, and subcutaneous injection), vaginally, rectally, and transdermally.

In some embodiments, the formulations are administered transdermally which includes all methods of administration across the surface of the body and the inner linings of body passages including epithelial and mucosal tissues. Such administration may be in the form of a lotion, cream, colloid, foam, patch, suspension, or solution.

Oral formulations containing the present polymorph can comprise any conventionally used oral forms, including tablets, capsules, buccal forms, troches, lozenges and oral liquids, suspensions or solutions. Capsules may contain mixtures of the crystalline form I in the desired percentage together any other polymorph(s) of pipindoxifene hydrochloride or amorphous pipindoxifene hydrochloride. Capsules or tablets of the desired crystalline form of the desired percentage composition may also be combined with mixtures of other active compounds or inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc.

Tablet formulations can be made by conventional compression, wet granulation, or dry granulation methods and utilize pharmaceutically acceptable diluents (fillers), binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations used herein can utilize standard delay or time release formulations or spansules. Suppository formulations can be made from traditional materials, including cocoa butter, with or without the addition of waxes to alter the suppositories melting point, and glycerin. Water soluble suppository bases, such as polyethylene glycols of various molecular weights, can also be used.

Example excipient systems suitable for preparing formulations of the present polymorph include one or more fillers, disintegrants, and lubricants.

The filler component can be any filler component known in the art including, but not limited to, lactose, microcrystalline cellulose, sucrose, mannitol, calcium phosphate, calcium carbonate, powdered cellulose, maltodextrin, sorbitol, starch, or xylitol.

Disintegrants suitable for use in the present formulations can be selected from those known in the art, including pregelatinized starch and sodium starch glycolate. Other useful disintegrants include croscarmellose sodium, crospovidone, starch, alginic acid, sodium alginate, clays (e.g. veegum or xanthan gum), cellulose floc, ion exchange resins, or effervescent systems, such as those utilizing food acids (such as citric acid, tartaric acid, malic acid, fumaric acid, lactic acid, adipic acid, ascorbic acid, aspartic acid, erythorbic acid, glutamic acid, and succinic acid) and an alkaline carbonate component (such as sodium bicarbonate, calcium carbonate, magnesium carbonate, potassium carbonate, ammonium carbonate, etc.). The disintegrant(s) useful herein can comprise from about 4% to about 40% of the composition by weight, preferably from about 15% to about 35%, more preferably from about 20% to about 35%.

The pharmaceutical formulations can also contain an antioxidant or a mixture of antioxidants, such as ascorbic acid. Other antioxidants which can be used include sodium ascorbate and ascorbyl palmitate, preferably in conjunction with an amount of ascorbic acid. An example range for the antioxidant(s) is from about 0.5% to about 15% by weight, most preferably from about 0.5% to about 5% by weight.

The formulations described herein can be used in an uncoated or non-encapsulated solid form. In some embodiments, the pharmacological compositions are optionally coated with a film coating, for example, comprising from about 0.3% to about 8% by weight of the overall composition. Film coatings useful with the present formulations are known in the art and generally consist of a polymer (usually a cellulosic type of polymer), a colorant and a plasticizer. Additional ingredients such as wetting agents, sugars, flavors, oils and lubricants may be included in film coating formulations to impart certain characteristics to the film coat. The compositions and formulations herein may also be combined and processed as a solid, then placed in a capsule form, such as a gelatin capsule.

Pharmaceutical compositions of pipindoxifene hydrochloride can be formulated with steroidal estrogens, such as conjugated estrogens, USP. The amount of pipindoxifene hydrochloride used in the formulation can be adjusted according to the particular polymorph form or ratio of polymorph forms used, the amount and type of steroidal estrogen in the formulation as well as the particular therapeutic indication being considered. In general, the pipindoxifene hydrochloride of defined polymorphic composition ratio can be used in an amount sufficient to antagonize the effect of the particular estrogen to the level desired. The dose range of conjugated estrogens can be from about 0.3 mg to about 2.5 mg, about 0.3 mg to about 1.25 mg, or about 0.3 mg to about 0.625 mg. An example range for amount of pipindoxifene hydrochloride in a combination formulation is about 10 mg to about 40 mg. For the steroidal estrogen mestranol, a daily dosage can be from about 1 μG to about 150 μG, and for ethynyl estradiol a daily dosage of from about 1 μG to 300 μG can be used. In some embodiments, the daily dose is between about 2 μG and about 150 μG.

In order that the invention disclosed herein may be more efficiently understood, examples are provided below. It should be understood that these examples are for illustrative purposes only and are not to be construed as limiting the invention in any manner.

### EXAMPLES

### Example 1

### Preparation of Pipindoxifene Hydrochloride Monohydrate Form I

A one-liter 3-neck flask equipped with a mechanical stirrer, temperature probe, reflux condenser and nitrogen atmosphere was charged with 150 g pipindoxifene hydrochloride, 1035 g, 1312 mL prefiltered ethanol and 188 g of purified water. The mixture was heated to 78-80° C. over a minimum of 45 min to form a solution. The resulting solution was stirred at a moderate speed for 15 min at 80° C. the stirrer speed was decreased to 75 rpm and the solution was cooled to the range of 22-25° C. over five hours. Crystallization began at 65-67° C. The slurry was held at 22-25° C. for a minimum of one hour, then the solid was collected by filtration on a 12.5 cm Buchner funnel fitted with paper. The cake was washed with ethanol (118 g/150 mL, prefiltered and precooled to 10-15° C. The cake was then dammed until dripping stopped at which point it had a depth of 1.6 cm. The cake had a weight of 157 g. The product was dried in a vacuum oven at 40° C., 25 mm Hg for one hour. The product was then milled, and the milled product continued drying in a vacuum oven at 25-35° C., 25 mm Hg for 18 hours to a moisture level of 3.5 to 5.5%. A DSC scan revealed the polymorph (Form I) with a peak at 179° C. See Example 6 for DSC procedures. The product yield was 86%.

### Example 2

### Procedure for Preparation of Pipindoxifene Hydrochloride Monohydrate Form II from Form I

A one-liter 3-neck flask equipped with a mechanical stirrer, temperature probe, reflux condenser and nitrogen atmosphere was charged with a sample of 20 g of pipindoxifene hydrochloride form 1,280 mL ethanol and 120 mL of purified water. The material added to the flask showed a DSC peak at 188° C. indicative of form I. The mixture was heated to reflux temperature to dissolve the pipindoxifene. The mixture was then cooled to 22° C. over three hours and a visible slurry forms. The mixture was filtered and the precipitate was washed with 20 mL of cold ethanol. The product was dried in a vacuum oven at 40° C. for 2 hours and then for an additional 22 hrs at room temperature. A DSC scan revealed the new polymorph (form II) with a peak at 179° C. See Example 6 for DSC procedures. The product yield was 74%.

### Example 3

### Alternative Preparation of Pipindoxifene Hydrochloride Monohydrate Form II from Form I

The procedure of Example 2 was followed with the noted variations: Starting material was 5 g of the product from Example 1 added to a 30% water/ethanol mixture (30 mL water: 70 mL ethanol). The mixture was heated to reflux, followed by cooling to room temperature over three hours, and then held at room temperature an additional hour. After filtration and rinsing with cold ethanol, the product was dried at 40° C. for two hours.

Yield of a material conforming to a DSC trace indicative of polymorph form II was 71%.

### Example 4

### Preparation of Pipindoxifene Hydrochloride Monohydrate Form I or Conversion from Form II to Form I

The procedures described in Example 1 were carried out with the following variations to maximize the yield of polymorph form I. The recrystallization step yielded increased percentages of Form I as the amount of alcohol relative to water was increased. The use of 12.5% water in ethanol resulted in a form exhibiting a DSC graph conforming with that shown in FIG. 4 with a melting point peak at 189° C. As the amount of ethanol relative to water decreased to levels approaching 2:1 v/v, the DSC curve shifted to the lower melting point of 180° C., indicating the predominance of polymorphic form II.

### Example 5

### X-Ray Powder Diffraction (XRPD)

XRPD analyses were carried out on a (Scintag X2) X-ray powder diffractometer using Cu K α radiation. The instrument was equipped with tube power, and amperage was set at 45 kV and 40 mA. The divergence and scattering slits were set at 1° and the receiving slit was set at 0.2 mm. A theta-two theta continuous scan at 3°/min (0.4 sec/0.02° step) from 3 to 40° 2θ was used.

### Example 6

### Differential Scanning Calorimetry (DSC)

DSC measurements were carried out in both sealed pan and vented pan at a scan rate of 10° C./min from 25° C. to 200° C. under nitrogen purge using a Pyris I DSC from Perkin-Elmer.

Various modifications of the invention, in addition to those described herein, will be apparent to those skilled in the art from the foregoing description. Such modifications are also intended to fall within the scope of the appended claims. Each reference, including all patents, patent applications, and journal literature, cited in the present application is incorporated herein by reference in its entirety.

What is claimed is:

1. A crystalline polymorph (form I) of pipindoxifene hydrochloride monohydrate having a powder X-ray diffraction pattern comprising characteristic peaks, in terms of 20, at about 21.2° and about 24.3°.

2. The polymorph of claim 1 wherein said powder X-ray diffraction pattern further comprises characteristic peaks, in terms of 20, at about 15.1° and about 19.2°.

3. The polymorph of claim 1 wherein said powder X-ray diffraction pattern comprises at least 5 characteristic peaks, in terms of 20, selected from about 13.9°, about 15.1°, about 18.1°, about 19.2°, about 21.2°, about 22.5°, about 24.3°, and about 26.4°.

4. The polymorph of claim 1 having a powder X-ray diffraction pattern substantially as shown in FIG. 2.

5. The polymorph of claim 1 having a differential scanning calorimetry trace showing maxima at about 145° C. and about 190° C.

6. The polymorph of claim 1 having a differential scanning calorimetry trace substantially as shown in FIG. 4.

7. A composition comprising the polymorph of claim 1.

8. The composition of claim 7 wherein at least about 50% by weight of total pipindoxifene hydrochloride monohydrate in said composition is present as said polymorph.

9. The composition of claim 7 wherein at least about 70% by weight of total pipindoxifene hydrochloride monohydrate in said composition is present as said polymorph.

10. The composition of claim 7 wherein at least about 80% by weight of total pipindoxifene hydrochloride monohydrate in said composition is present as said polymorph.

13. The composition of claim 7 wherein at least about 90% by weight of total pipindoxifene hydrochloride monohydrate in said composition is present as said polymorph.

14. The composition of claim 7 wherein at least about 95% by weight of total pipindoxifene hydrochloride monohydrate in said composition is present as said polymorph.

15. The composition of claim 7 wherein at least about 97% by weight of total pipindoxifene hydrochloride monohydrate in said composition is present as said polymorph.

16. The composition of claim 7 wherein at least about 98.0% by weight of total pipindoxifene hydrochloride monohydrate in said composition is present as said polymorph.

17. The composition of claim 7 wherein at least about 99.0% by weight of total pipindoxifene hydrochloride monohydrate in said composition is present as said polymorph.

18. A composition comprising the polymorph of claim 1 and a pharmaceutically acceptable carrier.

19. A composition consisting essentially of pipindoxifene hydrochloride monohydrate wherein at least 95% by weight of said pipindoxifene hydrochloride monohydrate is present in said composition as the polymorph of claim 1.

20. A composition consisting essentially of pipindoxifene hydrochloride monohydrate wherein at least 97% by weight of said pipindoxifene hydrochloride monohydrate is present in said composition as the polymorph of claim 1.

21. A composition consisting essentially of pipindoxifene hydrochloride monohydrate wherein at least 98.0% by weight of said pipindoxifene hydrochloride monohydrate is present in said composition as the polymorph of claim 1.

22. A composition consisting essentially of pipindoxifene hydrochloride monohydrate wherein at least 99.0% by weight of said pipindoxifene hydrochloride monohydrate is present in said composition as the polymorph of claim 1.

23. A composition comprising the polymorph of claim 1 and one or more steroidal estrogens.

24. A composition according to claim 23 wherein said steroidal estrogen component comprises conjugated estrogens.

25. A method of preparing pipindoxifene hydrochloride polymorphic form I comprising:
a) dissolving pipindoxifene hydrochloride in a solvent mixture comprising an alcohol, water, and optionally an ether; and
b) precipitating form I from said solvent mixture.

26. The method of claim 25 wherein said alcohol comprises ethanol.

27. The method of claim 25 wherein the volume ratio of water to alcohol is less than about 1:5.

28. The method of claim 25 wherein the volume ratio of water to alcohol is about 1:5 to about 1:10.

29. The method of claim 25 wherein said precipitating is induced by cooling said solvent mixture.

30. The polymorphic form I of pipindoxifene hydrochloride prepared by the method of claim 25.

31. The polymorph of claim 1 prepared by the method comprising:
a) dissolving pipindoxifene hydrochloride in a solvent mixture comprising an alcohol, water, and optionally an ether; and
b) precipitating form I from said solvent mixture.

32. A method of converting pipindoxifene hydrochloride monohydrate form I into a form II, comprising recrystallizing said form I from a solvent mixture comprising water and ethanol, wherein the volume ratio of water to ethanol is less than about 1:5.

33. A method of treating a mammal having a disease or syndrome associated with estrogen deficiency or excess of estrogen comprising administering to said mammal a therapeutically effective amount of a polymorph of claim 1.

34. A method of treating a mammal having a disease or disorder associated with proliferation or abnormal development of mammary tissues comprising administering to said mammal a therapeutically effective amount of a polymorph of claim 1.

35. A method of lowering cholesterol in a mammal comprising administering to said mammal a therapeutically effective amount of a polymorph of claim 1.

36. A method of inhibiting bone loss in a mammal comprising administering to said mammal a therapeutically effective amount of a polymorph of claim 1.

37. A method of treating breast cancer in a mammal comprising administering to said mammal a therapeutically effective amount of a polymorph of claim 1.

38. A method of treating a postmenopausal woman for one or more vasomotor disturbances comprising administering to said postmenopausal woman a therapeutically effective amount of a polymorph of claim 1.

39. The method of claim 38 wherein the vasomotor disturbance is hot flush.
